(19) United States
(12) Patent Application Publication
(10) Pub. No.: US 2011/0155136 A1
(43) Pub. Date: Jun. 30, 2011

(54) Gas Inspiratory and Expiratory Device and Respiratory Mask Having the Same

(75) Inventor: Gary C.J. LEE, I-Lan (TW)

(73) Assignee: GaleMed Xiamen Co., Ltd, Xiamen (CN)

(21) Appl. No.: 12/977,152

(22) Filed: Dec. 23, 2010

(30) Foreign Application Priority Data
Dec. 25, 2009 (CN) 200910259768.X
Dec. 28, 2009 (TW) 098145299

(51) Int. Cl. A61M 16/06; A61M 16/20

(52) U.S. Cl. 128/205.24; 128/205.25

(57) ABSTRACT

A gas inspiratory and expiratory device for a respiratory mask includes a tubular housing having top and bottom ends, an air chamber formed between the bottom and top ends, a vent hole for fluid communication of the air chamber with ambient atmosphere, and a passage hole for fluid communication of the air chamber with a CO₂ measuring apparatus. The top end is open to permit fluid communication of the air chamber with an internal portion of the respiratory mask. An inner tube is disposed within the air chamber, and includes an air inlet end disposed at the bottom end of the tubular housing, and an air supply end extending externally of the top end of the tubular housing.

### CROSS-REFERENCE TO RELATED APPLICATION

This application claims priority of Chinese Application No. 200910259768.X, filed on Dec. 25, 2009.

### BACKGROUND OF THE INVENTION

1. Field of the Invention

This invention relates to a gas inspiratory and expiratory device, and more particularly to a gas inspiratory and expiratory device that can separate flow of supplied oxygen and flow of CO₂ gas exhaled by a patient and a respiratory mask having the gas inspiratory and expiratory device.

2. Description of the Related Art

A conventional face mask assembly, as disclosed in U.S. Pat. No. 7,004,168, includes a face mask, a gas sample cell connected to a bottom end of the face mask, and an oxygen delivery housing connected to a bottom end of the gas sample cell. The oxygen delivery housing is used for coupling with an oxygen supply source. Oxygen provided by the oxygen supply source passes consecutively through the oxygen delivery housing, the gas sample cell, and the face mask to be inhaled by the patient. A CO₂ gas sensor may be connected to the gas sample cell to monitor the patient's breathing. Since the oxygen provided by the oxygen supply source and the CO₂ gas exhaled by the patient both flow through the gas sample cell, the CO₂ gas exhaled by the patient is affected by the oxygen provided by the oxygen supply source. This results in the CO₂ gas sensor measuring mixed gas (including undischarged gas from previous exhalation of the patient) instead of CO₂ gas only so that the CO₂ gas cannot be measured effectively and accurately.

### SUMMARY OF THE INVENTION

Therefore, the main object of the present invention is to provide a gas inspiratory and expiratory device for a respiratory mask. By supplying oxygen through an air flow channel for inhalation of a patient, air exhaled by the patient can flow to an air chamber and vent out through a unidirectional valve, so that the supplied oxygen and the air exhaled by the patient (including gas from previous exhalation) will not affect each other. Because of the relation between the expiration pressure and the weight of airflow, oxygen gas can flow into the mask body and the air exhaled by the patient can flow downward to a tubular housing of the gas inspiratory and expiratory device. Hence, the concentration value of CO₂ gas exhaled by the patient can be accurately measured.

Another object of the present invention is to provide a respiratory mask which has the aforesaid gas inspiratory and expiratory device.

The purpose of the present invention and the solution to the conventional technical problems are achieved through employment of the below technical means. According to one aspect of this invention, a gas inspiratory and expiratory device for a respiratory mask comprises a tubular housing and an inner tube. The tubular housing has a bottom end, a top end for connection with the respiratory mask, an air chamber formed between the bottom and top ends, a vent hole for fluid communication of the air chamber with ambient atmosphere, and a passage hole for fluid communication of the air chamber with a CO₂ measuring apparatus. The top end is open to permit fluid communication of the air chamber with an internal portion of the respiratory mask. The inner tube is disposed within the air chamber, and includes an air inlet end disposed at the bottom end of the tubular housing, and an air supply end extending externally of the top end of the tubular housing. According to another aspect of this invention, a respiratory mask comprises a mask body having a through hole, and a gas inspiratory and expiratory device including a tubular housing and an inner tube. The tubular housing has a bottom end, a top end for connection with the mask body, an air chamber formed between the bottom and top ends, an vent hole for fluid communication of the air chamber with ambient atmosphere, and a passage hole for fluid communication of the air chamber with a CO₂ measuring apparatus. The top end is open to permit fluid communication of the air chamber with an internal portion of the mask body. The inner tube is disposed within the air chamber, and includes an air inlet end disposed at the bottom end of the tubular housing, and an air supply end extending externally of the top end of the tubular housing.

Through the aforesaid technical means, the advantages and efficacy of the gas inspiratory and expiratory device of this invention resides in the fact that through the tubular housing that defines the air chamber, through the air supply end of the inner tube disposed in proximity to the patient's nose, and through cooperation of the inner tube and tubular connecting portion to define the air flow channel, oxygen supplied by the air supply tube can flow into the mask body through the air flow channel for patient's inhalation and will not affect the air exhaled by the patient. The exhaled air can flow into the air chamber through the top end of the surrounding wall. A portion of the exhaled air is vented through the vent hole, and the remaining portion thereof flows through the passage hole for measuring the concentration of the CO₂ gas. Through such a configuration, the CO₂ measuring apparatus can accurately measure the concentration of CO₂ gas exhaled by the patient. Further, through the configuration of the unidirectional valve, air exhaled by the patient can be vented to reduce the possibility of the patient repeatedly inhaling the air from the air chamber, thereby enhancing the concentration of oxygen gas and preventing the measurement of concentration of the CO₂ gas from being affected.

### BRIEF DESCRIPTION OF THE DRAWINGS

Other features and advantages of the present invention will become apparent in the following detailed description of the preferred embodiment of the invention, with reference to the accompanying drawings, in which:

FIG. 1 is a schematic side view of a respiratory mask according to the preferred embodiment of this invention;

FIG. 2 is an exploded partial sectional view of the preferred embodiment;

FIG. 3 is a schematic front view of a gas inspiratory and expiratory device of the preferred embodiment;

FIG. 4 is a view similar to FIG. 1, but illustrating a valve diaphragm of a unidirectional valve being pushed to deform by air from an air chamber for opening gradually a vent hole;

FIG. 5 illustrates how a portion of air exhaled by a patient may flow through a connector, a filter device, and a coupling tube into a CO₂ gas sensor;

FIG. 6 is a view similar to FIG. 2, but illustrating an alternative form of the gas inspiratory and expiratory device of the preferred embodiment; and

FIG. 7 is a view similar to FIG. 1, but illustrating an alternative form of the respiratory mask of this invention.

### DETAILED DESCRIPTION OF THE PREFERRED EMBODIMENT

The above-mentioned and other technical contents, features, and effects of this invention will be clearly presented from the following detailed description of a preferred embodiment in coordination with the reference drawings. Through description of the concrete implementation method, the technical means employed and the effectiveness to achieve the predetermined purposes of the present invention will be thoroughly and concretely understood. However, the enclosed drawings are used for reference and description only, and are not used for limiting the present invention.

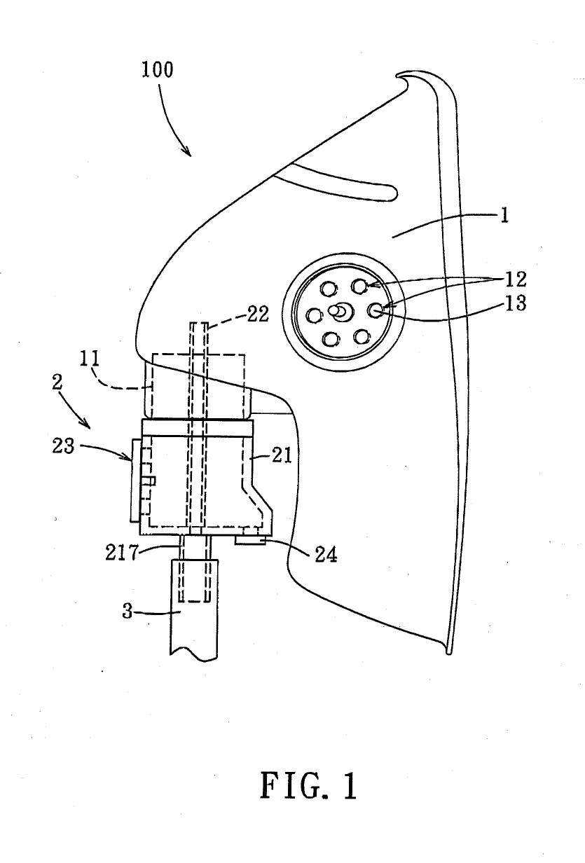

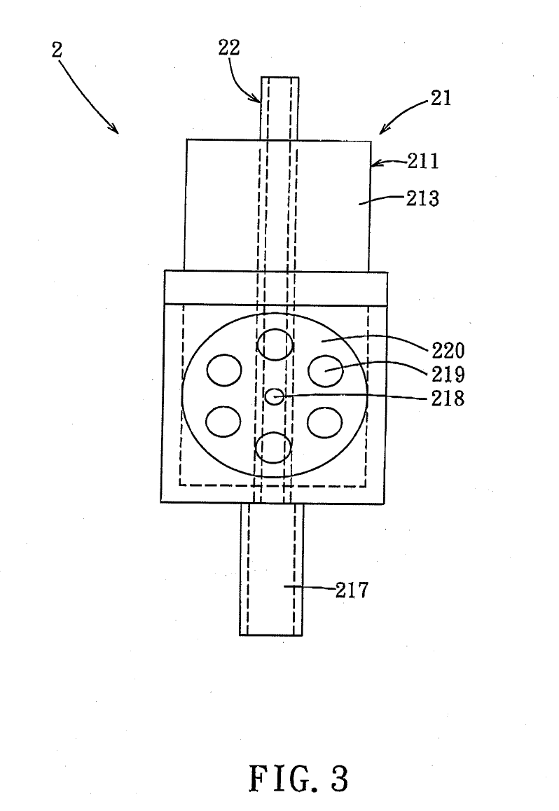

Referring to FIGS. 1 to 5, a respiratory mask 100 according to the preferred embodiment of the present invention comprises a mask body 1 for covering a patient's face, and a gas inspiratory and expiratory device 2 connected to a bottom end of the mask body 1. The gas inspiratory and expiratory device 2 may be connected to a respiratory machine (not shown) through an air supply tube 3, so that oxygen gas provided by the respiratory machine can pass through the air supply tube 3 and the gas inspiratory and expiratory device 2 into the mask body 1 for inhalation of the patient.

The gas inspiratory and expiratory device 2 includes a tubular housing 21 having a bottom end 214, and a top end 215 inserted into a through hole 11 of the mask body 1 that is proximate to the bottom end thereof. Preferably, the tubular housing 21 is interferencely fitted to the through hole 11 so that the tubular housing 21 cannot be easily detached from the mask body 1. The tubular housing 21 includes a bottom wall 212 at the bottom end 214, a surrounding wall 213 extending upwardly from an outer periphery of the bottom wall 212, and a tubular connecting portion 217 projecting downwardly from the bottom end 214. The bottom wall 212 and the surrounding wall 213 cooperatively define an air chamber 216 formed between the top and bottom ends 215, 214 of the tubular housing 21. The top end of the tubular housing 21 is open to permit fluid communication of the air chamber 216 with an internal portion of the mask body 1. Through such a configuration, air exhaled by the patient can flow into the air chamber 216.

The gas inspiratory and expiratory device 2 further includes an inner tube 22 disposed within the air chamber 216 and connected to and extending upwardly from the bottom wall 212. The inner tube 22 and the tubular connecting portion 217 are aligned to each other. The bottom wall 212 has a through hole 2120 interconnecting the tubular connecting portion 217 and the inner tube 22. In this embodiment, the bottom wall 212, the inner tube 22, and the tubular connecting portion 217 are formed as a one piece body. The inner tube 22 includes an air inlet end 221 disposed at the bottom end 214 of the tubular housing 21, and an air supply end 222 opposite to the air inlet end 221. The tubular connecting portion 217 communicates fluidly with the inner tube 22 through the air inlet end 221 so that the inner tube 22 and the tubular connecting portion 217 cooperatively define an air flow channel 223. Through such a configuration, oxygen gas supplied by the air supply tube 3 can flow into the mask body 1 through the air flow channel 223 for inhalation of the patient. Most preferably, the air supply end 222 of the inner tube 22 extends externally of the top end 215 of the tubular housing 21, and is proximate to the patient's nose (not shown). The more proximate the air supply end 222 is to the patient's nose the better. Hence, oxygen gas that flows through the air supply end 222 can be directly inhaled by the patient's nose.

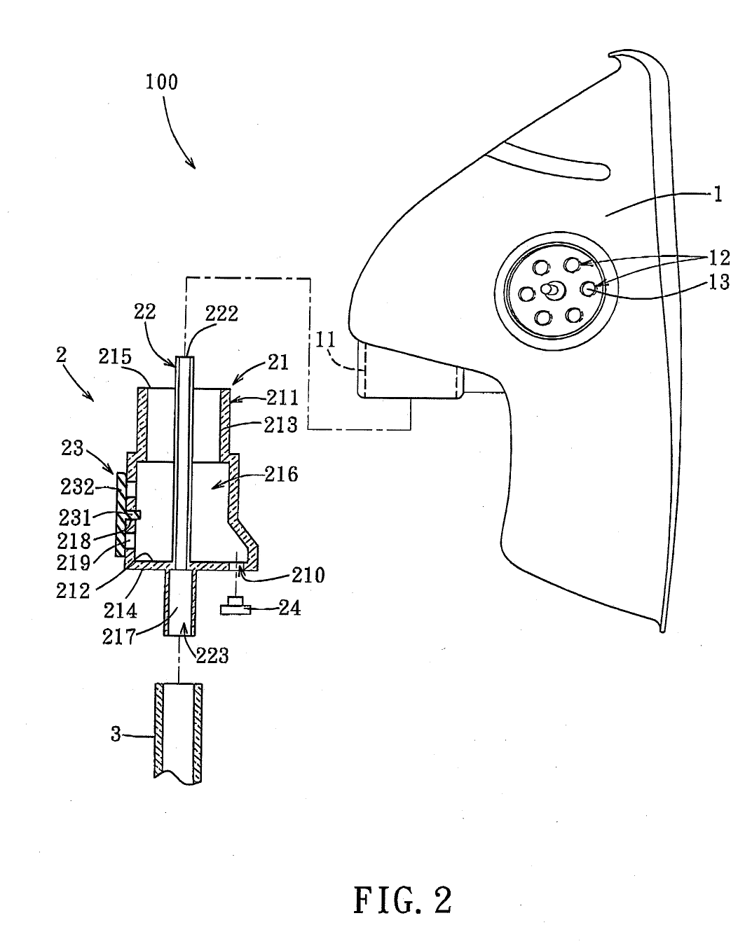

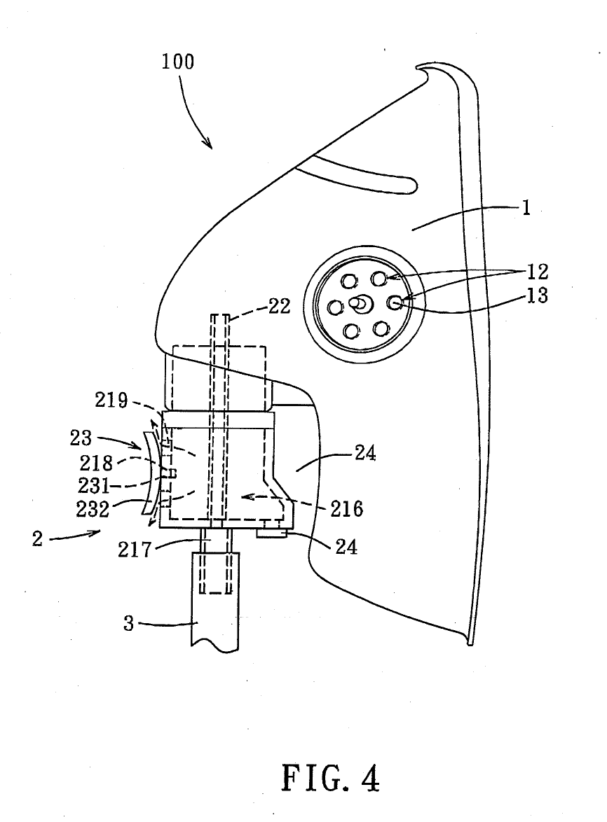

The gas inspiratory and expiratory device 2 further includes a unidirectional valve 23 provided on the tubular housing 21. The unidirectional valve 23 includes a valve diaphragm 232, and a plug 231 projecting from the valve diaphragm 232 into a mounting hole 218 in the surrounding wall 213. The plug 231 is interferencely fitted to the mounting hole 218 so that the plug 231 cannot be easily detached from the surrounding wall 213. The valve diaphragm 232 is used to close a plurality of vent holes 219 in the surrounding wall 213. The vent holes 219 permit fluid communication of the air chamber 216 with ambient atmosphere. Since the valve diaphragm 232 is made of a flexible material, such as rubber or silicone, during the patient's exhalation, air exhaled by the patient flows into the air chamber 216 through the top end 215 of the surrounding wall 213. The valve diaphragm 232 is pushed by the exhaled air to deform and open, as shown in FIG. 4, to vent air from the air chamber 216, so that patient's inhaling of the air from the air chamber 216 can be minimized. During inhalation, the flexible valve diaphragm 232 is restored to its original position shown in FIG. 2 to close the vent holes 219. Preferably, the surrounding wall 213 has a flat surface 220, so that the valve diaphragm 232 can abut flatly against the flat surface 220 to close tightly the vent holes 219.

To ensure that the amount of air inhaled by the patient during inhalation is sufficient to prevent the mask body 1 from producing depressions, the mask body 1 is provided with a plurality of air inlet holes 12, and a unidirectional air inlet valve diaphragm 13 provided on an inner face thereof to close the air inlet holes 12. The unidirectional air inlet valve diaphragm 13 is made of a flexible material, such as rubber or silicone. When an inhalation force of the patient reaches a definite degree, the unidirectional air inlet valve diaphragm 13 will deform and open to permit entry of external air into the mask body 1 through the air inlet holes 12. As such, the external air can mix with the oxygen gas that flows into the mask body 1 through the air supply end 222 for inhalation of the patient. The valve diaphragm 232 closes the vent holes 219 to provide a high concentration of oxygen gas to a patient, and the air inlet holes 12 can prevent insufficient input of oxygen gas to the patient.

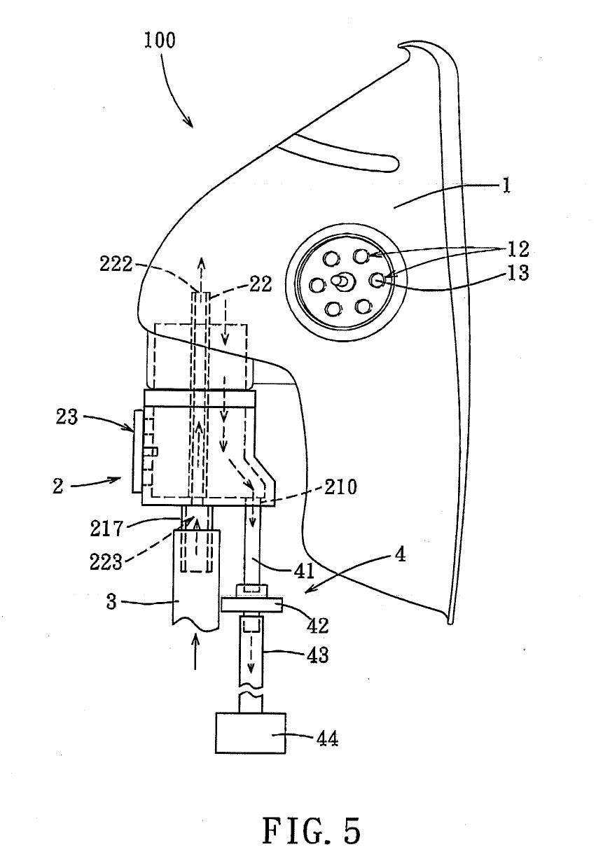

With reference to FIGS. 2 and 5, the tubular housing 21 is further provided with a passage hole 210 formed in the bottom end 214 for fluid communication of the air chamber 216 with ambient atmosphere. The passage hole 210 extends through the bottom wall 212. A caregiver may selectively cover the passage hole 210 using a cap 24. When the caregiver desires to measure the concentration of CO₂ gas exhaled by the patient, the cap 24 is removed, so that a CO₂ measuring apparatus 4 may be connected to the passage hole 210. The CO₂ measuring apparatus 4 includes a connector 41 having one end connected to the passage hole 210, a filter device 42 connected to the other end of the connector 41, a coupling tube 43 connected to the filter device 42, and a CO₂ concentration measuring device 44 connected to the coupling tube 43. In this embodiment, because the air supply end 222 is proximate to the patient's nose, the oxygen gas supplied by the air supply tube 3 can flow into the mask body 1 through the air flow channel 223 to be directly inhaled by the patient's nose, and will not affect the air exhaled by the patient. Air exhaled by the patient can flow into the air chamber 216 through the top end 215 of the surrounding wall 213. A portion of the exhaled air is vented through the vent holes 219, and the remaining portion thereof flows into the CO₂ concentration measuring device 44 through the connector 41, the filter device 42, and the coupling tube 43.

The filter device 42 filters the moisture and foreign matter of the air exhaled by the patient. Since the oxygen gas flowing into the mask body 1 through the air flow channel 223 will not affect the air exhaled by the patient, the CO₂ concentration measuring device 44 can accurately measure the concentration of the CO₂ gas exhaled by the patient. Further, since CO₂ is heavier than oxygen, and the exhaled air of the patient continuously flow downward, through the provision of the passage hole 210 in the bottom wall 212, air can flow effectively into the connector 41.

Moreover, because an outer diameter of the surrounding wall 213 of the tubular housing 21 is large, the air exhaled by the patient can easily flow into the air chamber 216 through the top end 215, preventing accumulation of the CO₂ gas exhaled by the patient in the mask body 1 to be repeatedly inhaled by the patient and affecting the measurement of concentration of the CO₂ gas.

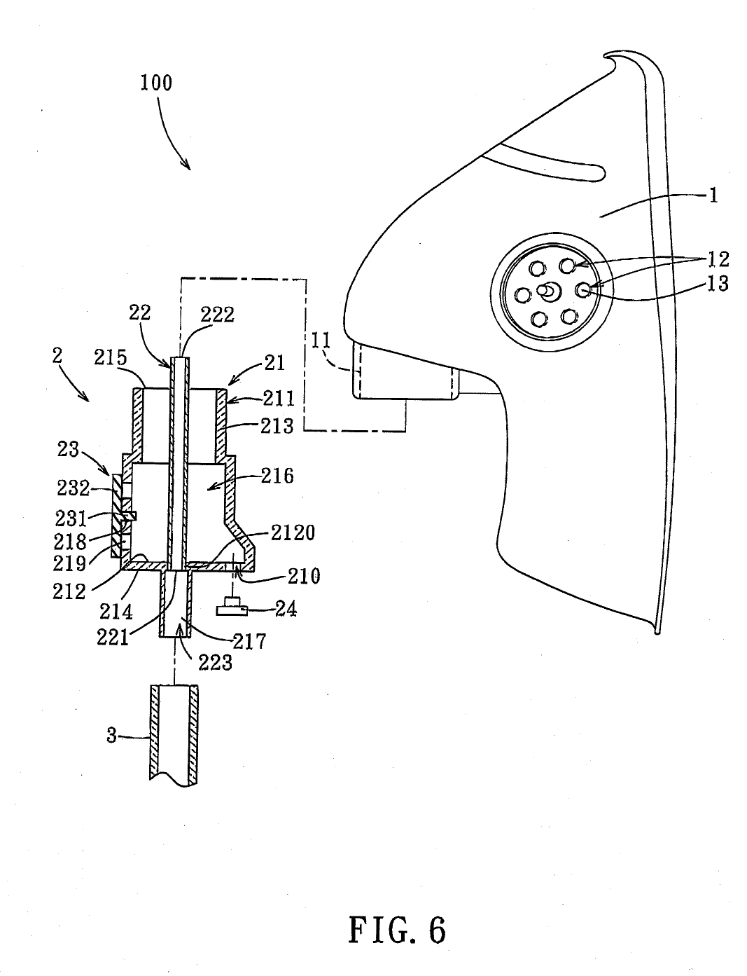

It is worth mentioning that in an alternative embodiment, as shown in FIG. 6, the inner tube 22 may be inserted into the through hole 2120 in the bottom wall 212 so that the inner tube 22 communicates fluidly and directly with the tubular connecting portion 217. Further, the gas inspiratory and expiratory device 2 may be dispensed with the tubular connecting portion 217, in this case, through the insertion of the small-diameter air supply tube 3 into the air inlet end 221 of the inner tube 22, supply of oxygen gas into the inner tube 22 can be similarly and effectively achieved.

From the aforesaid description, the respiratory mask 100 of the present invention, through the tubular housing 21 that defines the air chamber 216, through the air supply end 222 of the inner tube 22 being proximate to the patient's nose, and through the inner tube 22 and the tubular connecting portion 217 cooperatively defining the air flow channel 223, oxygen gas supplied by the air supply tube 3 can flow into the mask body 1 through the air flow channel 223 to be directly inhaled by the patient's nose, and will not affect the air exhaled by the patient. The air exhaled by the patient can flow into the air chamber 216 through the top end 215 of the surrounding wall 213 with a portion of the air being vented through the vent hole 219 and the remaining portion thereof flowing through the passage hole 210 to proceed with the measuring of the concentration of the CO₂ gas. As such, the CO₂ concentration measuring device 44 can accurately measure the concentration of the CO₂ gas exhaled by the patient. Further, through the configuration of the unidirectional valve 23, the air exhaled by the patient can be vented to reduce the possibility of the patient repeatedly inhaling the air from the air chamber 216.

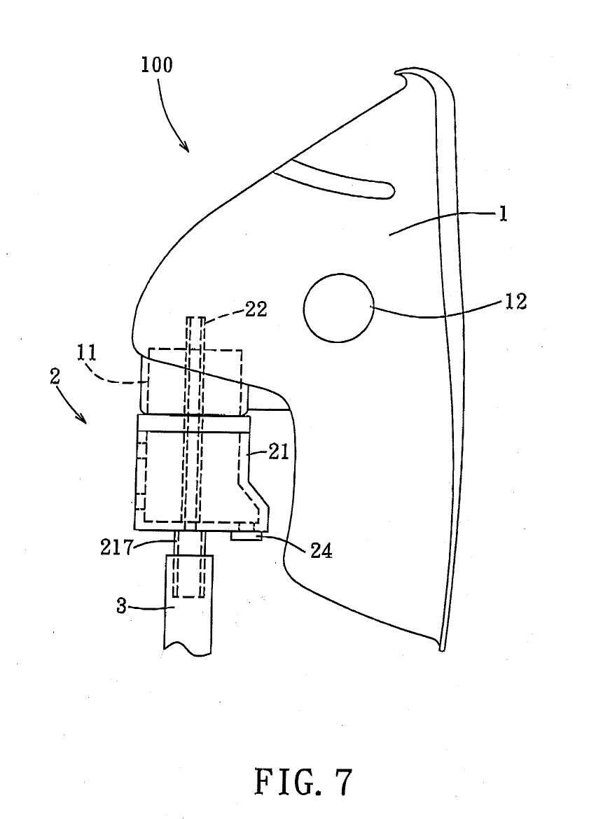

In an alternative embodiment of the respiratory mask 100 of the present invention, as shown in FIG. 7, the unidirectional air inlet valve diaphragm 13 provided on the mask body 1 and the unidirectional valve 23 provided on the tubular housing 21 are dispensed with. A similar effect as described above may be achieved. The respiratory mask 100 shown in FIG. 7 is suitable for used by a patient that does not require a high concentration of oxygen gas. Although the concentration of oxygen gas is reduced, it will not affect the measurement of concentration of the CO₂ gas. While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

I claim:

1. A gas inspiratory and expiratory device for a respiratory mask, comprising:
a tubular housing having a bottom end, a top end for connection with the respiratory mask, an air chamber formed between said bottom and top ends, a vent hole for fluid communication of said air chamber with ambient atmosphere, and a passage hole for fluid communication of said air chamber with a CO₂ measuring apparatus, said top end being open to permit fluid communication of said air chamber with an internal portion of the respiratory mask; and
an inner tube disposed within said air chamber and including an air inlet end disposed at said bottom end of said tubular housing, and an air supply end extending externally of said top end of said tubular housing.

2. The gas inspiratory and expiratory device of claim 1, wherein said tubular housing includes a bottom wall at said bottom end, and a tubular connecting portion projecting downwardly from said bottom end, said inner tube being connected to and extending upwardly from said bottom wall, said tubular connecting portion and said inner tube being aligned with each other, said bottom wall having a through hole interconnecting said tubular connecting portion and said inner tube.

3. The gas inspiratory and expiratory device of claim 2, wherein said bottom wall, said inner tube, and said tubular connecting portion are formed as a one piece body.

4. The gas inspiratory and expiratory device of claim 2, wherein said inner tube is inserted into said through hole in said bottom wall.

5. The gas inspiratory and expiratory device of claim 2, wherein said tubular housing further includes a surrounding wall extending upwardly from an outer periphery of said bottom wall and having said vent hole, said surrounding wall further having a mounting hole, said gas inspiratory and expiratory device further comprising a unidirectional valve provided on said tubular housing to close said vent hole, said unidirectional valve being openable to vent air from said air chamber through said vent hole and including a valve diaphragm to close said vent hole, and a plug projecting from said valve diaphragm into said mounting hole.

6. The gas inspiratory and expiratory device of claim 5, wherein said surrounding wall further has a flat surface, said valve diaphragm abutting against said flat surface to close said vent hole.

7. A respiratory mask comprising:
a mask body; and
a gas inspiratory and expiratory device including a tubular housing having a bottom end, a top end for connection with said mask body, an air chamber formed between said bottom and top ends, an vent hole for fluid communication of said air chamber with ambient atmosphere, and a passage hole for fluid communication of said air chamber with a CO₂ measuring apparatus, said top end being open to permit fluid communication of said air chamber with an internal portion of said mask body; and
an inner tube disposed within said air chamber and including an air inlet end disposed at said bottom end of said tubular housing, and an air supply end extending externally of said top end of said tubular housing.

8. The respiratory mask of claim 7, wherein said tubular housing includes a bottom wall at said bottom end, and a tubular connecting portion projecting downwardly from said bottom end, said inner tube being connected to and extending upwardly from said bottom wall, said tubular connecting portion and said inner tube being aligned with each other, said bottom wall having a through hole interconnecting said tubular connecting portion and said inner tube.

9. The respiratory mask of claim 8, wherein said bottom wall, said inner tube, and said tubular connecting portion are formed as a one piece body.

10. The respiratory mask of claim 8, wherein said inner tube is inserted into said through hole in said bottom wall.

11. The respiratory mask of claim 8, wherein said tubular housing further includes a surrounding wall extending upwardly from an outer periphery of said bottom wall and having said vent hole, said surrounding wall further having a mounting hole, said respiratory mask further comprising a unidirectional valve provided on said tubular housing to close said vent hole, said unidirectional valve being openable to vent air from said air chamber through said vent hole and including a valve diaphragm to close said vent hole, and a plug projecting from said valve diaphragm into said mounting hole.

12. The respiratory mask of claim 11, wherein said surrounding wall further has a flat surface, said valve diaphragm abutting against said flat surface to close said vent hole.

13. The respiratory mask of claim 7, wherein said mask body further includes an air inlet hole, and a unidirectional air inlet valve diaphragm for covering said air inlet hole, said unidirectional air inlet valve diaphragm being openable for permitting entry of external air into said mask body through said air inlet hole.

14. The respiratory mask of claim 7, wherein said mask body has a through hole communicating with ambient atmosphere.
